(19) United States
(12) Patent Application Publication
(10) Pub. No.: US 2025/0143542 A1
(43) Pub. Date: May 8, 2025

(54) SYSTEMS AND METHODS FOR ENDOSCOPIC IMAGE DEPTH ESTIMATION

(71) Applicant: Verathon Inc., Bothell, WA (US)

(72) Inventor: Riley Cambon, Vancouver (CA)

(21) Appl. No.: 18/909,026

(22) Filed: Oct. 8, 2024

Related U.S. Application Data
(60) Provisional application No. 63/596,346, filed on Nov. 6, 2023.

(51) Int. Cl. A61B 1/00; A61B 1/05; A61B 1/06; A61B 1/267

(52) U.S. Cl. CPC A61B 1/000094; A61B 1/000095; A61B 1/00057; A61B 1/05; A61B 1/0676; A61B 1/2676

(57) ABSTRACT

An endoscopic camera captures an image of a scene that is illuminated by the light source. A processor performs image linearization for the image based on a stored gamma curve, estimates tissue colors in the image based on stored tissue color estimation data, and corrects for incident light intensity based on the estimated tissue color. The processor also corrects for light beam pattern intensity, based on a calibration image, to obtain corrected light intensity for the image. The processor generates a depth map for the image based on the corrected light intensity and provides a measurement of an object in the image based on the depth map.

### CROSS-REFERENCE TO RELATED APPLICATION

This application claims priority under 35 U.S.C. § 119, based on U.S. Provisional Patent Application No. 63/596,346 filed Nov. 6, 2023, titled “Systems and Methods for Endoscopic Image Depth Estimation,” the disclosure of which is hereby incorporated by reference.

### BACKGROUND

This present disclosure relates to video medical device procedures and, more particularly, to an endoscopic procedures with an image capturing component for allowing accurate examination of a patient's internal passages.

Endoscopic devices, such as laryngoscopes and bronchoscopes, with video cameras have made it possible to display an image of a patient's internal anatomy from a remote position. Such endoscopic devices may enable detection of biological features within the patent. Clinicians have found that there is clinical value in being able to measure the size of biological features within the patent, such as tumors, stones, and the prostate. However, obtaining accurate measurements in three dimensions from a two-dimensional image produced by an endoscope remains a challenge.

### BRIEF DESCRIPTION OF THE DRAWINGS

FIGS. 1A and 1B illustrates a video endoscope system consistent with implementations described herein;

FIG. 2 is a block diagram functional components implemented in the endoscopic system of FIG. 1A;

FIG. 3 is a block diagram of logical components of a depth image processing module, according to an implementation;

FIG. 4 includes images illustrating conversion from an original captured image to a linearized/grayscale output;

FIG. 5 includes images illustrating conversion from an original captured image to an image with estimated tissue color and conversion from the estimated tissue color to a grayscale image with color intensity correction;

FIG. 6 includes images illustrating a corrected intensity image from application of a calibration image to a grayscale image with color intensity correction;

FIG. 7 includes images illustrating generation of a depth map from a corrected intensity image;

FIG. 8 is an image illustrating an example user interface and measurement geometry for a depth image, according to an implementation;

FIG. 9 is a flow diagram of a process for estimating image depth in an endoscopic image, according to an exemplary implementation; and

FIG. 10 is a block diagram illustrating components of an endoscope controller.

### DETAILED DESCRIPTION OF EMBODIMENTS

The following detailed description refers to the accompanying drawings. The same reference numbers in different drawings may identify the same or similar elements. Also, the following detailed description does not limit the invention.

Systems and methods described herein calculate image depth for endoscopic applications and provide three-dimensional (3D) measurements of biological features using standard endoscope hardware. A video-based endoscope and system includes video capture components that capture images and transmit the captured images to a remote monitor viewable by the user.

An endoscope described herein may include both single-use (i.e., disposable) and reusable endoscopes that include image capturing and lighting elements. During and after insertion of the endoscope into the patient's anatomy, images obtained from the image capturing elements are conveyed to a video monitor viewable by the endoscope user via a data cable. Consistent with embodiments described herein, the endoscope, the data cable, and the remote video monitor may each include logic components configured to enable image data to be exchanged between the image capturing element and the video monitor in an efficient and optimized manner.

To measure the size of an object in an image (e.g., a photograph), an accurate depth map is typically required. The depth of an object in an image can be extracted through various methods, but most methods are difficult to implement in endoscopic applications as they require additional hardware. Current clinical methods for measuring the size of biological features include visual estimation, surgical removal, and 3D scanning devices, such as MRI, CT, and ultrasound.

There are also other 3D scanning techniques that are currently used in a variety of industries. For example, Structured Light Scanning (SLS) operates on projecting a known light pattern onto a surface from a known angle and position relative to the camera. This method requires additional light emitting hardware and optics at a predefined distance between the camera source and projector source. This process has been described for endoscopic applications but requires costly optics that take up space. Stereoscopic Video is operated by analyzing two synchronized video inputs that are spatially separated by a known distance. This method requires an additional camera and processing hardware to synchronize the two video feeds. Video Analysis uses an input video to create a depth map of an environment. This method requires knowledge of the camera's position in space which can be accomplished using an additional hardware such as an accelerometer.

Each of the above techniques require additional equipment that adds cost and takes up space in the constrained environment of the distal tip of the endoscope. Systems and methods for endoscopic depth estimation are needed that can avoid the additional cost and space of conventional depth estimation techniques.

Systems and methods described herein use existing endoscope hardware to calculate image depth for endoscopic applications and provide three-dimensional (3D) measurements of biological features. Properties of the LED illumination source and the camera used in an endoscope are applied to calculate an estimated depth at each point in an image obtained by the endoscope. The systems and methods analyze the level of reflected light off biological tissue, correct for estimated tissue color, and use the LED characteristics to estimate depth.

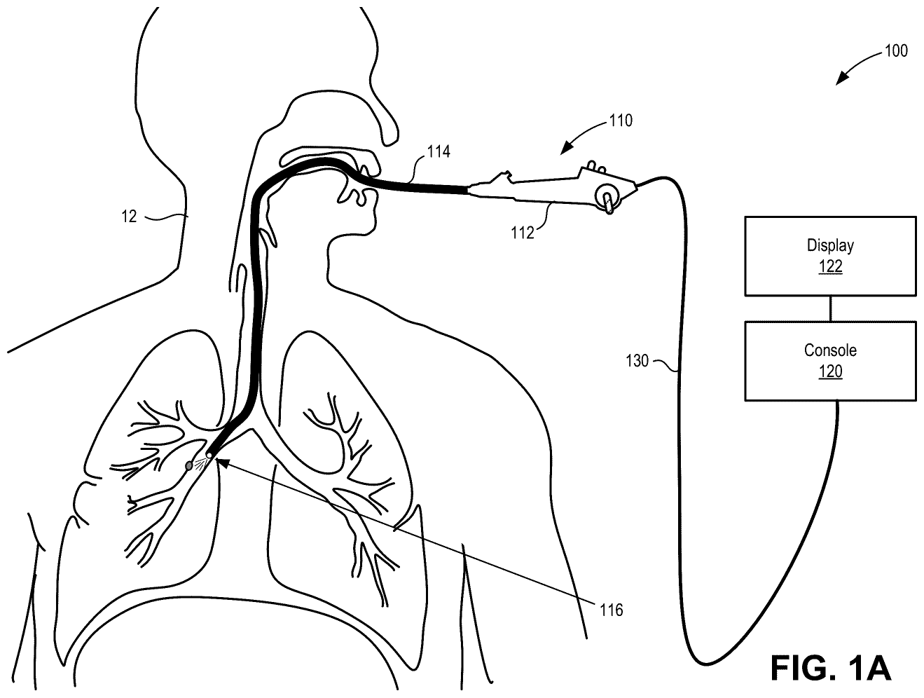

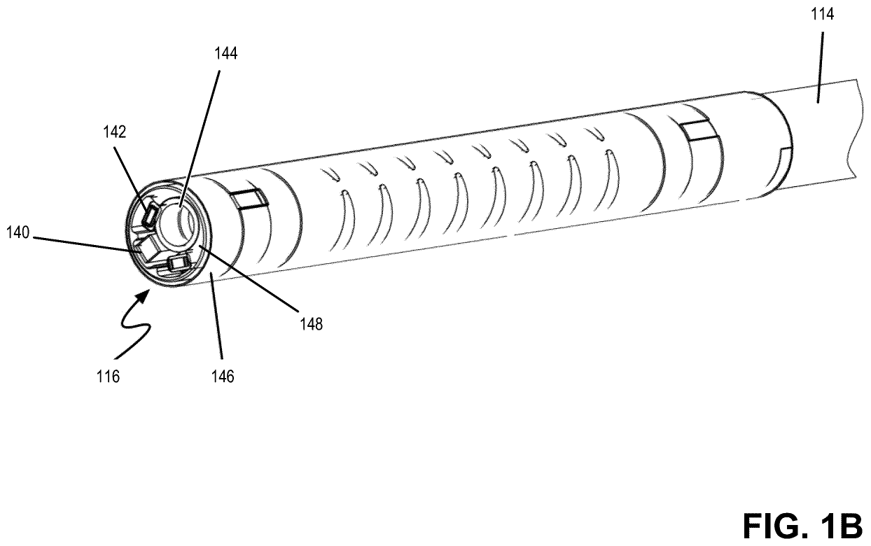

FIG. 1A illustrates a video endoscope system 100 consistent with implementations described herein. Endoscopic system 100 includes an endoscope 110 and a console 120 connected by a data cable 130.

A cross-sectional view of the upper body of a patient 12 is shown with a side view of the endoscope 110 (e.g., a bronchoscope) delivered to the bronchus of the patient 12. Endoscope 110 includes a handle 112 connected to an insertion shaft 114 that is inserted into the patient 12 and directed to a region of interest. For example, a distal tip 116 of the endoscope 110 may be positioned in proximity to one or more bronchial lymph nodes. The endoscope 110 may be connected to console 120, which may include (or be connected to) a display 122.

FIG. 1B is an isometric view of the distal tip 116 portion of endoscope shaft 114 consistent with implementations described herein. As shown, distal tip 116 includes a camera module 140, a light source 142, and a working channel 144 within an outer wall 146 that forms a main lumen 148. Main lumen 148 is sized to accommodate the internal components of shaft 114, which include camera module 140, light source 142, working channel 144, control wires, and any wiring necessary for the operation of camera module 140 and light source 142.

Camera module 140 includes a camera (e.g., video camera, still camera, or another image capturing device) to obtain images of an area inside the patient 12. Light source 142 includes light-emitting diodes (LEDs) or other lighting devices to illuminate an object (e.g., an object to be measured) within an anatomical region in the field of view of the camera lens. According to an implementation, illumination in the scene is provided only by the light source 142 of endoscope 110. No additional light sources are present in the scene. Furthermore, the object being measured is within the camera's depth of view (e.g., 5-50 millimeters). Camera module 140 and light source 142 are sufficiently sealed within distal tip 116 to prevent moisture or fluids from reaching the internal optical electronics of the camera and light source.

In some implementations, camera module 140 and light source 142 may be formed as part of a circuit board assembly, such as a printed circuit board assembly (PCBA), flexible printed circuit board assembly (FPCBA), or rigid flexible printed circuit board assembly (RFPCBA) (not shown). In one implementation, the PCBA (or FPCBA/RFPCBA) may be configured to couple camera module 140 and light source 142 to a data interface assembly in handle 112 via wires (not shown) that extend the length of shaft 114. In alternative embodiments, camera module 140 and light source 142 may be coupled directly to wires and may not be integrated with or coupled to a PCBA.

As shown in FIG. 1A, endoscope 110 may communicate with console 120 via a wired connection, such as via data cable 130. In other implementations, endoscope 110 may communicate with console 120 via a wireless connection (e.g., Bluetooth, WiFi, etc.). In each case, console 120 is connected to and/or includes display 122 to allow a user to view images obtained by camera 140, and/or to allow operational interaction with respect to the user during operation of endoscope 110. In some embodiments data cable 130 may include one or more components of camera module 140, such as a serializer component. In such an embodiment, the cable 130 may further include one or more logical components configured to identify when an endoscope has been connected, which endoscope has been connected, and to negotiate with video monitor 106 to determine which of the cable 130 and the display 122 have the most up-to-date camera settings for use during image capture.

Display 122 may include an output display/screen, such as a liquid crystal display (LCD), light emitting diode (LED) based display, or other type of display that presents text and/or image data to a user. For example, display 122 may display two-dimensional or three-dimensional images of a selected anatomical region. As described further herein, display 122 may also provide a depth map of a captured environment.

Images obtained from camera module 140 are conveyed to display 122 (e.g., via data cable 130 and/or console 120) and viewable by the endoscope user (not shown). Collected images may also be provided to console 120 for processing. Using the known characteristics of camera module 140 and light source 142 of endoscope 110, console 120 can create a depth map of the image without requiring additional hardware. Once a depth map is calculated, console 120 can provide the image for three-dimensional measurements.

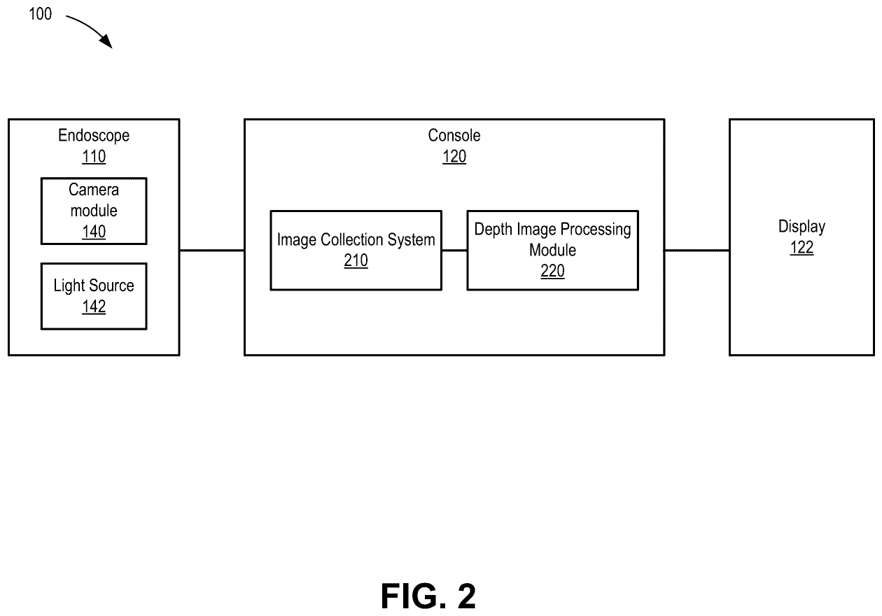

FIG. 2 is a block diagram of functional components implemented in endoscopic system 100 in accordance with an implementation. System 100 includes endoscope 110 and console 120. Endoscope 110 includes camera module 140 and light source 142. Console 120 includes an image collection system 210 and depth image processing 220.

Camera module 140 may include a camera and associated logic to obtain two-dimensional digital images. Camera module 140 may include automatic gain control (AGC) and/or automatic exposure control (AEC), which is designed to improve image quality by automatically boosting the gain and increasing the exposure in low light images so that objects can be seen more clearly and reduce the gain and decrease the exposure in bright images to avoid the subject of the image from being washed out or blurry. In some implementations, upper or lower limits for AGC and/or AEC in camera module 140 may be modified for different environments inside the body. Camera module 140 may provide captured (e.g., still) images to image collection system 210, for example. Along with sending each captured image to image collection system 210, camera module 140 may include image metadata, such as a camera gain value (e.g., an ACG value) and a light intensity value (an AEC value) at the time of capturing the image.

Light source 142 may include, for example, one or more light emitting diodes (LEDs) to illuminate a scene viewed by camera module 140. For example, light source 142 may illuminate an anatomical region inside a patient (e.g., patient 12) within the field of view of a camera lens. The location/orientation of light source 142 within distal tip 116 may produce a light distribution pattern (or beam pattern) with consistent characteristics at different known distances.

Image collection system 210 may include an interface and memory to receive and store images and associated metadata from camera module 140. According to implementations described herein, image collection system 210 may provide captured images and image data to depth image processing module 220 for estimating image depth.

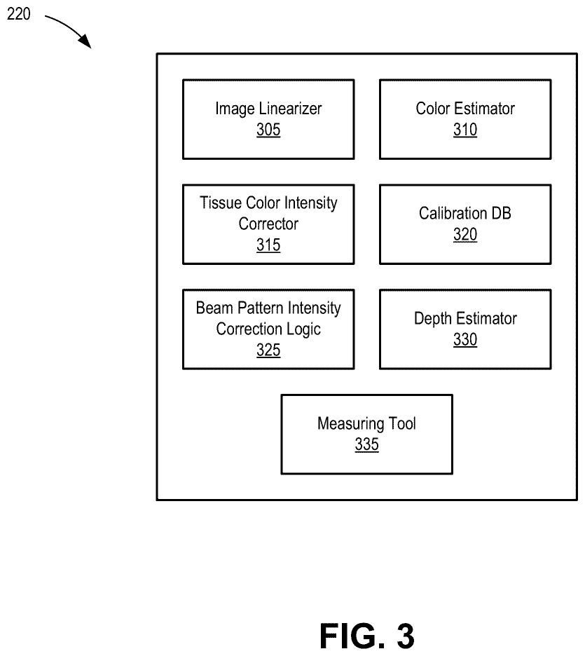

Depth image processing module 220 may include one or more processors to receive images from image collection system 210 and perform image conversions to create a depth map of the image. Depth image processing module 220 is described further below in connection with FIG. 3, for example.

Display 122 may present images from image collection system 210 and/or depth image processing module 220. In some implementations, display 122 may include a video monitor with a control pad. In such implementations, practitioners (e.g., medical personnel) may interface with display 122 during use to initiate image capture by camera module 140, freeze a particular frame, or adjust certain settings. As briefly described above, in some implementations display 122 may be integrated within console 120 to which endoscope is coupled via data cable 130. In other implementations, display 122 may be separate from console or in addition to a display provided within console 122, such as a remote viewing monitor, a streaming video client device, etc.

FIG. 3 is a block diagram of logical components of depth image processing module 220. As shown in FIG. 3, depth image processing 220 may include an image linearizer 305, a color estimator 310, tissue color intensity corrector 315, a calibration database (DB) 320, beam pattern intensity correction logic 325, a depth estimator 330, and a measuring tool 335.

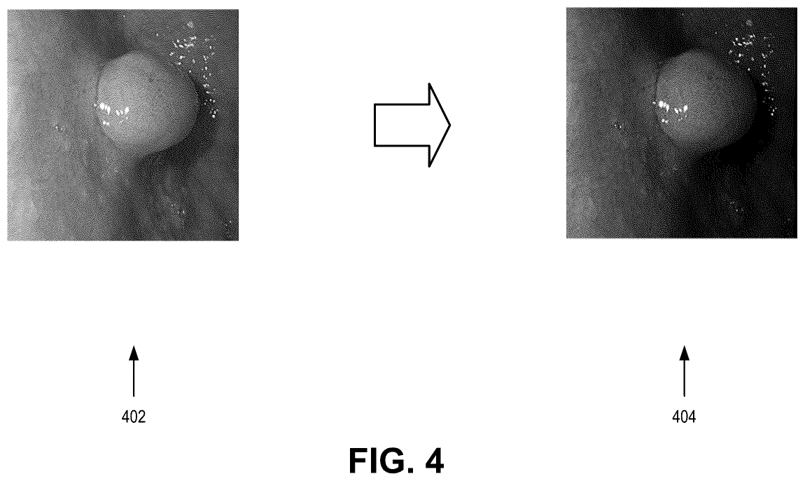

Image linearizer 305 may obtain an image from image collection system 210. Image linearizer 305 may convert the image into a grayscale image and linearize the image using a known gamma curve of the camera. The gamma curve may define a logarithmic relationship of how smoothly the camera images transition from black to white. In one implementation, the gamma curve may be defined during a camera tuning stage and hard coded in image linearizer 305 prior to an image capture procedure. After performing a color-to-grayscale conversion, image linearizer 305 may encode the non-linear values from the image the camera obtains into a linear relationship (e.g., based on the gamma curve). FIG. 4 illustrates the conversion from an original captured image 402 to a linearized/grayscale output 404 that image linearizer 305 may perform.

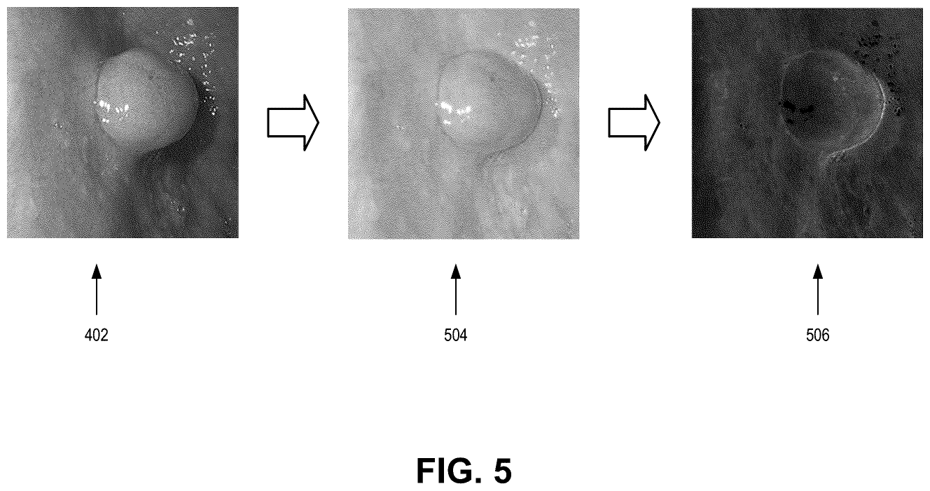

Color estimator 310 may estimate the color of tissue in the scene of the original image received from image collection system 210. In one implementation, color estimator 310 may work in parallel with image linearizer 305. As different tissue colors will have different light reflectivity levels, color estimator 310 may match the original image color pixels to known color options for an atomical region. In one implementation, color estimator 310 may select an initial tissue color based on a best estimate to known tissue colors stored in a local database. For example, color estimator 310 may include a lookup table to determine reflectivity for each pixel based on the tissue chromaticity. FIG. 5 illustrates the conversion from an original captured image 402 to an image with estimated tissue color 504 that may be performed by color estimator 310.

Tissue color intensity corrector 315 may apply light intensity corrections to color-adjusted images from color estimator 310. For example, using the estimated pixel color from color estimator 310, tissue color intensity corrector 315 may calculate the incident light intensity as a percentage of the reflected light. Tissue color intensity corrector 315 may then calculate a reflectivity correction factor based on the estimated tissue color. The reflectivity correction factor compensates for color variation in features, such as vascularization and cartilage. Tissue color intensity corrector 315 may apply the reflectivity correction factor to image pixels with the estimated tissue color to provide color intensity correction. FIG. 5 illustrates the conversion from the image with estimated tissue color 504 to a grayscale image with color intensity correction 506 that may be performed by tissue color intensity corrector 315.

Calibration database 320 may store calibration information for camera module 140 and/or light source 142. For example, calibration database 320 may store beam pattern calibration images for light source 142. The intensity of a light source beam pattern is not typically uniform across the camera field of view. Calibration images of the beam pattern of light source 142 at a known distance may be stored in beam calibration database 320 and applied to compensate for the non-uniformity of the LED beam pattern particular to light source 142. For example, different calibration images may be stored for different distances/depths. In one aspect, beam pattern calibration involves capturing a beam projection onto a surface at a known distance and known reflectivity (e.g., the beam projected onto a surface 30 millimeters away where the surface has 80% reflectance). The gain and exposure levels of camera module 140 may also be captured at this time to calibrate the absolute intensity of the calibrated beam pattern. In some implementations, calibration database 320 may also store gamma curve data for camera module 140 and tissue color estimation data for known biological tissues, as described further herein.

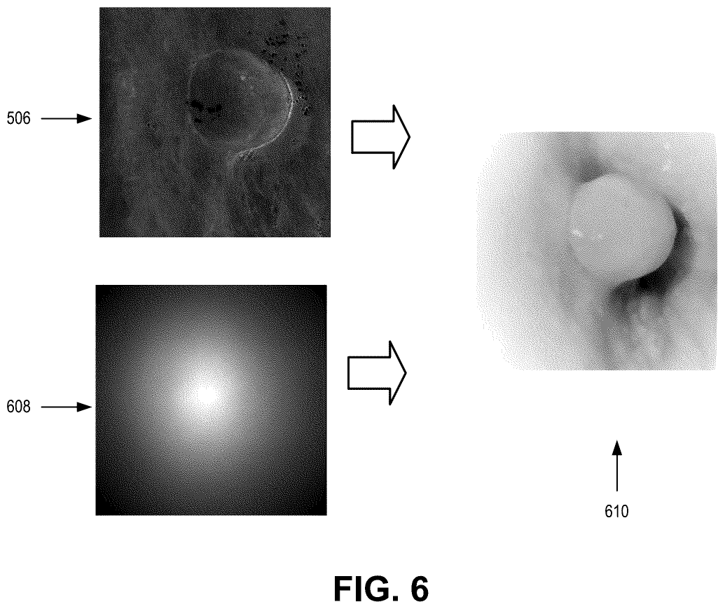

Beam pattern intensity correction logic 325 may apply calibration images from calibration database 320 to the color intensity corrected image from color estimator 310 and calibrated beam image from tissue color intensity corrector 315. Beam pattern intensity correction logic 325 uses a calibration image of the beam pattern at a known distance to compensate for LED non-uniformity. For example, as shown in FIG. 6, beam pattern intensity correction logic 325 may use a calibration image 608 to apply pixel-by-pixel adjustments to the grayscale image with color intensity correction 506. The resultant image may be a corrected intensity image 610.

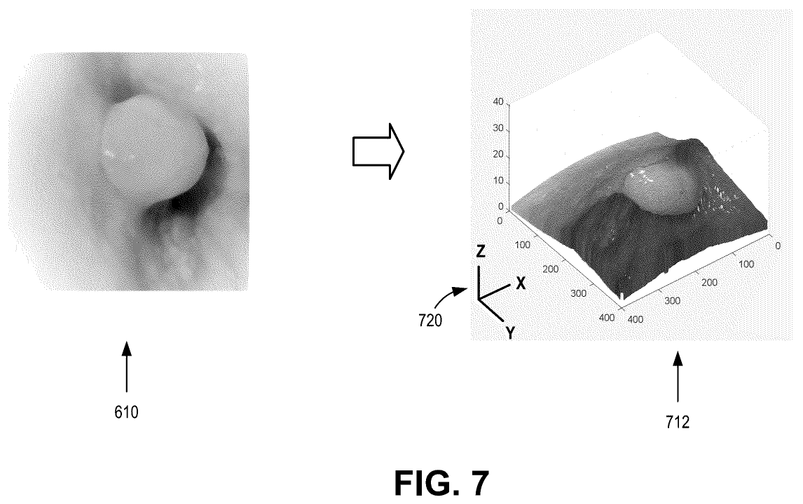

Depth estimator 330 may receive processed images from beam pattern intensity correction logic 325 and generate a depth map for the image based on the corrected light intensity for the image (e.g., corrected intensity image 610). According to an implementation, depth estimator 330 may apply the inverse square law of light intensity to determine depth on a pixel-by-pixel basis. The inverse square law states that the intensity of light radiating from a point source is inversely proportional to the square of the distance from the source. In addition to the inverse square law, depth estimator 330 may also correct for the incidence angle of the light source 142 using shape from shading techniques. For example, the angle of incidence to the surface may be estimated by taking an image gradient at multiple image resolutions. The surface normal from each image resolution may then be averaged to get a final estimated surface gradient. The output of this process is a depth map where each pixel in corrected intensity image 610 has a calculated depth value. For example, as shown in FIG. 7, depth estimator 330 may use corrected intensity image 610 to map the depth (and other dimensions) of the captured image. According to an implementation, depth estimator 330 may apply a depth map to the original image (e.g., image 402) to create a depth map image 712. In one implementation, the orientation of original image 402 may be altered to superimpose a scene/object on a depth map in a three-axis orientation, such as axis 720.

Measuring tool 335 may provide a user interface to permit a practitioner to obtain three-dimensional measurements of objects in a depth map image. For example, measuring tool 335 may receive marking input on display 122 from a user via an input tool (e.g., a mouse, pen, touch screen, etc.). The marking input may indicate, for example, a beginning point, ending point, and a direction (e.g., a X, Y, or Z direction of axis of 720) for a measurement. Measuring tool 335 may calculate a dimension based on the user input and depth map image. Although described herein in connection with depth image processing module 220, in other implementations, measuring tool 335 may be included as a separate logical component in console 120 or display 122.

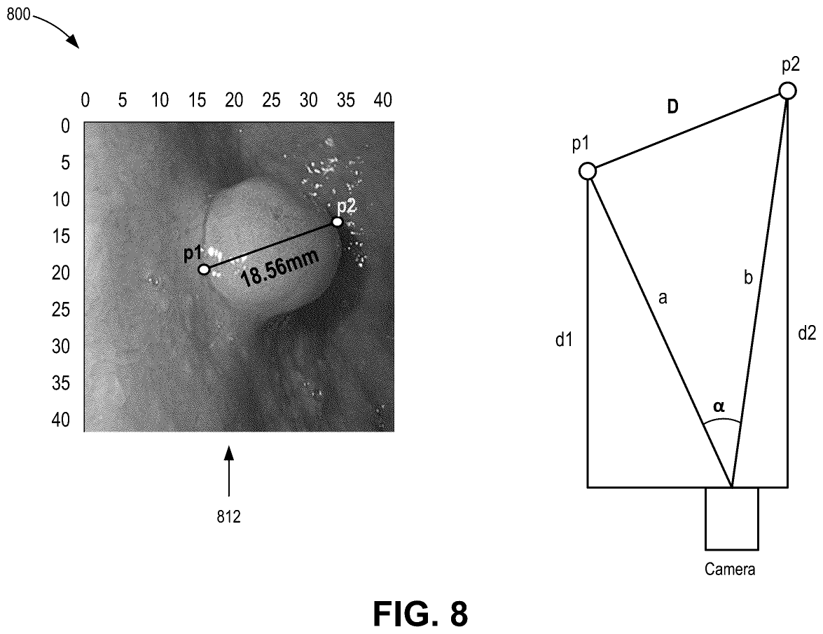

FIG. 8 illustrates an example user interface 800 and measurement geometry for measuring a depth image using measuring tool 335, according to an implementation. Using a mouse, digital pen, or another input device, a user may mark a depth map image 812 (which may correspond to depth map image 712) with a beginning point, p1, and an ending point, p2. Measuring tool 335 may calculate a distance, D, between p1 and p2 based on geometry defined for the pixel at p1 and the pixel at p2 relative to camera module 140. As a simplified example, an angle between the two points, α, may be determined by the following equations:

$$α\_x = abs ( FOV ) * ( p 2 \_x - p1\_x )$$

$$α\_y = abs ( FOV ) * ( p 2 \_y - p1\_y )$$

$$α = sqrt ( α\_x \hat{} 2 + α\_y \hat{} 2 ) ,$$

where FOV is a dimension of the camera field of view. From angle α, the distance D may be determined as follows:

$$a = d 1 * cos ( α / 2 )$$

$$b = d 2 * cos ( α / 2 )$$

$$D = sq rt ( a \hat{} 2 + b \hat{} 2 - 2 * a * b * cos ( α ) ) .$$

The equations above a simplified for two-dimensions. Similar calculations may be used with respect to X- and Z-axis dimensions and/or Y- and Z-axis dimensions to identify a three-dimensional distance between points p1 and p2. As shown in FIG. 8, measuring tool 335 may present to the user a calculated value for D (e.g., “18.56 mm”) superimposed over depth map image 812 or at another portion of display 122 (not shown) relative to depth map image 812.

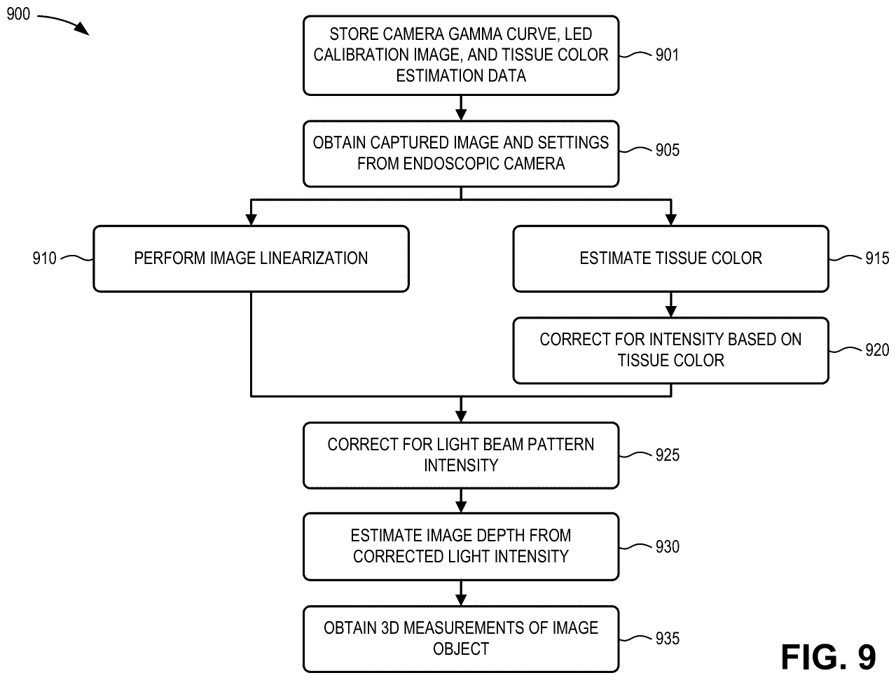

FIG. 9 is a flow diagram of a process 900 for estimating image depth in an endoscopic image. In one implementation, process 900 may be performed by console 120. In another implementation, process 900 may be performed by console 120 in conjunction with endoscope 110 and/or display 122.

Process 900 may include storing camera gamma curve, LED calibration images, and tissue color estimation data (block 901) and obtaining a captured image and settings from an endoscopic camera (block 905). For example, console 120 may store (e.g., in database 320) gamma curve data for camera module 140, calibration image of the beam pattern of light source 142 at a known distance, and tissue color estimation data. Endoscope 110 may use camera module 140 to capture an original image (e.g., image 402) inside a body (e.g., of patient 12) where only illumination from light source 142 is present. In another implementation, settings, such as camera gain and exposure levels, may also be obtained from the time the image is captured. The original image and settings may be provided to console 120 (e.g., image collection system 210) via data cable 130, for example.

Process 900 may further include performing image linearization (block 910). For example, console 120 (e.g., image linearizer 305) may convert the original captured image (e.g., image 402) into a grayscale image. The grayscale image may then be linearized based on the known gamma curve for camera 140 retrieved, e.g., from database 320.

Process 900 may also include estimating tissue colors (block 915) and correcting for incident light intensity (block 920). For example, console 120 (e.g., color estimator 310) may estimate the color of the tissue in the original image scene (e.g., image 402). Using the estimated pixel color, console 120 (e.g., tissue color intensity corrector 315) may correct for incident light intensity in the image based on the estimated tissue color. For example, tissue color intensity corrector 315 may calculate the incident light intensity as a percentage of the reflected light.

Process 900 may include correcting for light beam pattern intensity (block 925) and estimating an image depth from the reflected light intensity (block 930). For example, console 120 (e.g., beam pattern intensity correction logic 325) may apply calibration images from a memory (e.g., calibration database 320) to compensate for non-uniform light distribution from light source 142. Console 120 (e.g., depth estimator 330) may estimate distances for each pixel using, for example, the inverse square law of light intensity. To correct for the incidence angle of light on the measured surface, shape from shading techniques may be used to determine a final estimated surface gradient. Depth estimator 330 may generate, for example, a depth map where each pixel in the image has a calculated depth value.

Process 900 may further include obtaining three-dimensional measurements of an image object (block 935). For example, a user may use console 120 (e.g., measuring tool 335) in conjunction with display 122 to obtain three-dimensional measurements of an object, such as a biological feature, in the original image. In one implementation, the original image may be overlaid on a depth map to provide visual context for measurement.

FIG. 9 illustrates one example process of estimating image depth in an endoscopic image. According to other exemplary embodiments, console 120 may perform additional operations, fewer operations, and/or different operations than those illustrated and described. For example, console 120 may provide additional tools or perform additional measurements for an object. In another implementation, console 120 may subsequently transmit to or receive data (e.g., measurement data) to another device (e.g., a networked decide, a user device, etc.).

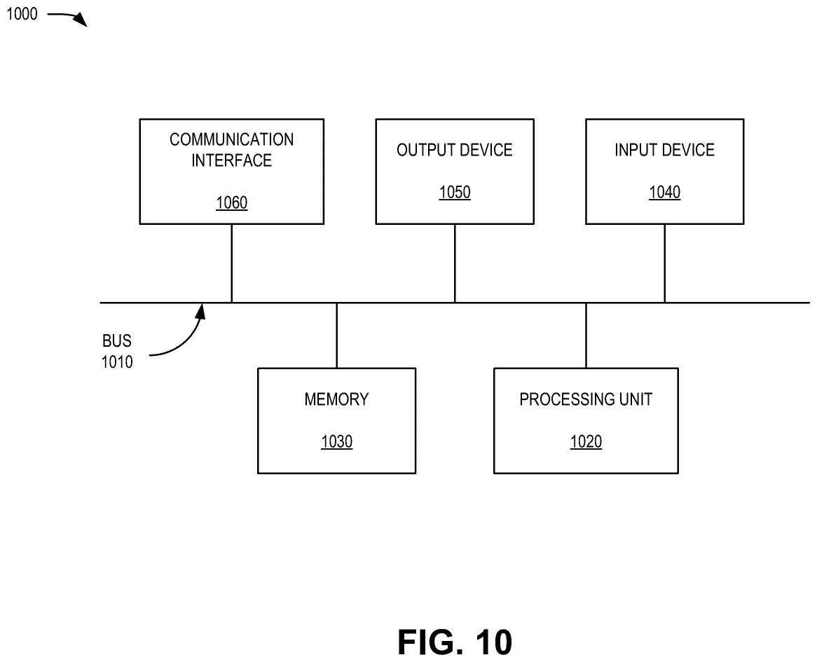

FIG. 10 is a diagram of exemplary components of a device 1000 that may correspond to console 120. As shown in FIG. 10, device 1000 may include a bus 1010, a processing unit 1020, a memory 1030, an input device 1040, an output device 1050, and a communication interface 1060.

Communication path or bus 1010 may provide an interface through which components of network device 1000 can communicate with one another.

Processing unit 1020 may include one or more processors or microprocessors that interpret and execute instructions, such as instructions for calculating image depth for endoscopic applications and providing three-dimensional measurements of biological features, as described herein. In other implementations, processing unit 1020 may be implemented as or include one or more application specific integrated circuits (ASICs), field programmable gate arrays (FPGAs), or the like.

Memory 1030 may include a random access memory (RAM) or another type of dynamic storage device that stores information and instructions for execution by processing unit 1020, a read only memory (ROM) or another type of static storage device that stores static information and instructions for the processing unit 1020, and/or some other type of magnetic or optical recording medium and its corresponding drive for storing information and/or instructions.

Input device 1040 may include a device that permits an operator to input information to device 1000, such as a keyboard, a keypad, a mouse, a pen, a microphone, one or more biometric mechanisms, and the like. Output device 1050 may include a device that outputs information to the operator, such as a display, a speaker, etc.

Communication interface 1060 may include a transceiver, such as a radio frequency transceiver, that enables device 1000 to communicate with other devices and/or systems. For example, communication interface 1060 may include mechanisms for communicating with other devices, such as other computing devices. Each of such other devices may include its respective communication interface 1060 to achieve such communication.

As described herein, device 1000 may perform certain operations in response to processing unit 1020 executing software instructions contained in a computer-readable medium, such as memory 1030. A computer-readable medium may include a tangible, non-transitory memory device. A memory device may include space within a single physical memory device or spread across multiple physical memory devices. The software instructions may be read into memory 1030 from another computer-readable medium or from another device via communication interface 1060. The software instructions contained in memory 1030 may cause processing unit 1020 to perform processes described herein. Alternatively, hardwired circuitry may be used in place of or in combination with software instructions to implement processes described herein. Thus, implementations described herein are not limited to any specific combination of hardware circuitry and software.

Although FIG. 10 shows exemplary components of device 1000, in other implementations, device 1000 may contain fewer, different, differently-arranged, or additional components than depicted in FIG. 10. In still other implementations, a component of device 1000 may perform one or more other tasks described as being performed by another component of device 1000.

Systems and methods are provided for calculating image depth for endoscopic applications and providing three-dimensional measurements of biological features. An endoscopic camera captures an image of a scene that is illuminated by the light source and provides the image to a console. A processor in the console performs image linearization for the image based on a stored gamma curve, estimates tissue colors in the image based on stored tissue color estimation data, and corrects for incident light intensity based on the estimated tissue color. The processor also corrects for light beam pattern intensity, based on a calibration image, to obtain corrected light intensity for the image. The processor generates a depth map for the image based on the corrected light intensity and provides a measurement of an object in the image based on the depth map.

The systems and methods may be performed using standard endoscope hardware, such as single-use endoscope hardware, to capture an image. In contrast, 3D scanning techniques are expensive to implement in single-use devices, and the additional hardware required for 3D scanning uses up space at the distal tip, which is already very limited. When applied, systems and methods described herein, for the measurement of biological features without needing specialized equipment, tools, or change in workflow.

As set forth in this description and illustrated by the drawings, reference is made to “an exemplary embodiment,” “an embodiment,” “embodiments,” etc., which may include a particular feature, structure or characteristic in connection with an embodiment(s). However, the use of the phrase or term “an embodiment,” “embodiments,” etc., in various places in the specification does not necessarily refer to all embodiments described, nor does it necessarily refer to the same embodiment, nor are separate or alternative embodiments necessarily mutually exclusive of other embodiment(s). The same applies to the term “implementation,” “implementations,” etc.

The foregoing description of embodiments provides illustration, but is not intended to be exhaustive or to limit the embodiments to the precise form disclosed. Accordingly, modifications to the embodiments described herein may be possible. For example, various modifications and changes may be made thereto, and additional embodiments may be implemented, without departing from the broader scope of the invention as set forth in the claims that follow. Also, while a series of blocks have been described with regard to FIG. 8 the order of the blocks and message/operation flows may be modified in other embodiments. Further, non-dependent blocks may be performed in parallel. The description and drawings are accordingly to be regarded as illustrative rather than restrictive.

The terms “a,” “an,” and “the” are intended to be interpreted to include one or more items. Further, the phrase “based on” is intended to be interpreted as “based, at least in part, on,” unless explicitly stated otherwise. The term “and/of” is intended to be interpreted to include any and all combinations of one or more of the associated items. The word “exemplary” is used herein to mean “serving as an example.” Any embodiment or implementation described as “exemplary” is not necessarily to be construed as preferred or advantageous over other embodiments or implementations.

Use of ordinal terms such as “first,” “second,” “third,” etc., in the claims to modify a claim element does not by itself connote any priority, precedence, or order of one claim element over another, the temporal order in which acts of a method are performed, the temporal order in which instructions executed by a device are performed, etc., but are used merely as labels to distinguish one claim element having a certain name from another element having a same name (but for use of the ordinal term) to distinguish the claim elements. Similarly, relative terms, such as “upper/lower”, “front/rear”, and “forward/backward” are used to depict relative positioning with respect to described components and do not refer to absolute or gravity-based relative positions. Embodiments described herein may be implemented in any suitable orientation.

Certain features described above may be implemented as “logic” or a “module” that performs one or more functions. This logic or module may include hardware, such as one or more processors, microprocessors, application specific integrated circuits, or field programmable gate arrays, software, or a combination of hardware and software.

No element, act, or instruction used in the description of the present application should be construed as critical or essential to the invention unless explicitly described as such.

What is claimed is:

1. A system comprising:
an endoscope including a camera module and a light source module, and
a console including a processor to:
receive, from the endoscope, an image of a scene, obtained by the camera module, that is illuminated by the light source;
perform image linearization for the image based on a stored gamma curve;
estimate tissue colors in the image based on stored tissue color estimation data;
correct the image for incident light intensity based on the estimated tissue color;
correct the image for light beam pattern intensity, based on a calibration image, to obtain corrected light intensity for the image;
generate a depth map for the image based on the corrected light intensity; and
provide a measurement of an object in the image based on the depth map.

2. The system of claim 1, wherein the processor is further configured to store in a memory of the console:
the gamma curve for the camera module, and
the calibration image for the light source module.

3. The system of claim 2, wherein the calibration image includes a beam pattern for the light source module at a known distance.

4. The system of claim 1, wherein, when performing the image linearization, the processor is further configured to:
convert the image from color to grayscale.

5. The system of claim 1, further comprising:
a data cable configured to transfer data between the endoscope and the console.

6. The system of claim 1, wherein, when generating the depth map, the processor is further configured to:
calculate a depth value for each pixel in the image.

7. The system of claim 1, wherein, when receiving the image of a scene, the processor is further configured to receive:
a camera gain value and a light intensity value at a time of capturing the image.

8. The system of claim 1, wherein the processor is further configured to store in a memory of the console:
the tissue color estimation data for known biological tissues.

9. The system of claim 1, wherein, when estimating the tissue colors, the processor is further configured to:
estimate a tissue color for each pixel in the image.

10. A method, comprising:
receiving, from an endoscopic camera, an image of a scene illuminated by the light source;
performing image linearization for the image based on a stored gamma curve;
estimating tissue colors in the image based on stored tissue color estimation data;
correcting the image for incident light intensity based on the estimated tissue color;
correcting the image for light beam pattern intensity, based on a calibration image, to obtain corrected light intensity for the image;
generating a depth map for the image based on the corrected light intensity; and
providing a measurement of an object in the image based on the depth map.

11. The method of claim 10, further comprising:
storing, in a memory, the gamma curve for the camera module and the calibration image for the light source module.

12. The method of claim 10, wherein the calibration image includes a beam pattern for the light source module at a known distance.

13. The method of claim 10, wherein performing the image linearization further comprises:
converting the image from color to grayscale.

14. The method of claim 10, wherein generating the depth map further comprises:
calculating a depth value for each pixel in the image.

15. The method of claim 10, wherein receiving the image of a scene further comprises:
receiving a camera gamma value and a light intensity value at a time the image was captured.

16. The method of claim 10, wherein estimating the tissue colors further comprises:
estimating a color for each pixel in the image.

17. A non-transitory computer-readable storage medium storing instructions executable by a processor of a network device, wherein the instructions are configured to:
receive, from an endoscopic camera, an image of a scene illuminated by the light source;
perform image linearization for the image based on a stored gamma curve;
estimate tissue colors in the image based on stored tissue color estimation data;
correct the image for incident light intensity based on the estimated tissue color;
correct the image for light beam pattern intensity, based on a calibration image, to obtain corrected light intensity for the image;
generate a depth map for the image based on the corrected light intensity; and
provide a measurement of an object in the image based on the depth map.

18. The non-transitory computer-readable storage medium of claim 17, wherein the instructions are further to:
store, in a memory, the gamma curve for the camera module, the calibration image for the light source module, and the tissue color estimation data for known biological tissues.

19. The non-transitory computer-readable storage medium of claim 17, wherein the instructions to generate the depth map, further comprise instruction to:
calculating a depth value for each pixel in the image.

20. The non-transitory computer-readable storage medium of claim 17, wherein the instructions to estimate the tissue colors, further comprise instruction to:
estimate a color for each pixel in the image.
